(19) United States
(12) Patent Application Publication
(10) Pub. No.: US 2006/0149228 A1
(43) Pub. Date: Jul. 6, 2006

(54) Device for dynamically stabilizing bones or bone fragments, especially thoracic vertebral bodies

(75) Inventors: Johannes Fridolin Schlapfer, Holstein (CH); Manuel Schan, Baselstrasse 13 (CH)

Correspondence Address: KNOBBE MARTENS OLSON & BEAR LLP, 2040 MAIN STREET, FOURTEENTH FLOOR, IRVINE, CA, 92614, US

(73) Assignee: Stratec Medical, Oberdorf (CH)

(21) Appl. No.: 10/542,646

(22) PCT Filed: May 5, 2004
(86) PCT No.: PCT/EP04/04775

§ 371 Date: Jul. 19, 2005

(30) Foreign Application Priority Data
Jun. 12, 2003 (DE) 103 26 517.1

(51) Int. Cl. A61F 2/30

(52) U.S. Cl. 606/61

(57) ABSTRACT

Apparatus for the dynamic stabilization of bone fragments, in particular spinal vertebrae (V), with at least one longitudinal support (11) that can be fixed to the vertebrae (V). The at least one longitudinal support (11) is so constructed that by application of a prespecified bending force it can be plastically deformed from a first stable shape state into a second, alternative stable shape state, but while in the first as well as in the second state remains flexible within predetermined limits. Preferably the longitudinal support (11) comprises a metallic core (12) surrounded by a plastic casing (13).

The present invention relates to an apparatus for the dynamic stabilization of bones or bone fragments, in particular spinal vertebrae, with at least one longitudinal support that can be fixed to the vertebrae.

The main indications for dynamic fixation, in particular when performed from the posterior aspect, are age- or disease-induced degeneration of structures in the spinal column as well as inflammation and/or injuries in the region of the intervertebral disk, the ligament apparatus, the facet joints and/or the subchondral bone.

Posterior dynamic fixation systems have the function of modifying the movement pattern in the affected spinal-column segment in such a way that the pains produced by chemical stimulation (nucleus material in contact with neural structures) and/or by mechanical stimulation (hypermobility) disappear, while the metabolism of the structures is preserved or restored.

Clinical experience with existing posterior dynamic fixation systems, such as are described for example in EP 0 669 109 B1 and in the manual entitled “Fixateur externe” (authors: B. G. Weber and F. Magerl, Springer-Verlag 1985, pp. 290-366), shows that a posterior dynamic fixation system is advantageous in being both flexible with respect to bending and stiff with respect to compression (buckling), shear forces and rotation. Thus such a system must be dimensioned so as to allow maximal deformation under flexion and also to resist the greatest possible force in the directions of buckling, shear and rotation. These conditions are in themselves contradictory, and in order to reconcile them the longitudinal supports are advantageously constructed of a biocompatible high-performance plastic material. Because such materials have a modulus of elasticity much lower than those of titanium and steel, these longitudinal supports can be made relatively thick in comparison to the steel and titanium versions in general clinical use, without any loss of flexibility; this is beneficial regarding their resistance to shear forces and buckling, as follows:
critical load for buckling: Pkr=const.*E*φ⁴
critical shear force: Qkr=const.*τₘₐₓ*φ²
critical bending: akr=const.*σₘₐₓ*1/E*1/φ

The above formulas show how the material properties, the E modulus and the diameter can be modified in order to be able to fulfill the various criteria regarding deformation and resistance.

The problem encountered when biocompatible high-performance plastic is used for the longitudinal supports is that such structures, in contrast to metallic longitudinal supports, can be permanently bent in situ only with considerable technical difficulty, e.g. by heating.

It is particularly important for longitudinal supports to be bendable in the case of posterior stabilization by way of pedicle screws, because when these are screwed into the vertebra by way of the pedicle, they often turn out to be incorrectly aligned on account of the anatomical situation. In order nevertheless to connect the longitudinal supports to the pedicle screws with the least possible tension, the shape of the supports must be adjusted to the position and orientation of the pedicle screws in situ. In the case of polyaxial pedicle screws the necessity of bending can be limited to one plane, whereas with monoaxial pedicle screws the longitudinal supports must be bent three-dimensionally.

Another embodiment of a dynamic fixation system is proposed in EP 0 690 701 B1. This system comprises a connecting rod that can be fixed at its ends to two adjacent vertebrae and that comprises a curved middle section, so that it is flexible within certain limits. In other respects the shape of this connecting rod cannot be altered.

The document WO 01/45576 A1 also proposes a dynamic stabilization system incorporating a longitudinal support, which here comprises two metallic end sections that can be fixed within complementary openings in the heads of two adjacent pedicle screws. Between the two end sections is disposed a linking element that is flexible in the long direction and preferably is made of flexible material. Both of the end sections of the longitudinal support are rigid. In addition to this linking element it is proposed that an elastic band be disposed between two pedicle screws, which extends parallel to the elastic linking element.

In this embodiment, again, the longitudinal extent of the linking element is prespecified by the manufacturer, and hence cannot be altered. Finally, reference should be made to the construction according to FR 2 799 949, which is characterized by the construction of the longitudinal support as a spring element, for example in the form of a leaf spring curved into a meander shape.

In the construction according to WO 98/22033 A1 the longitudinal support also comprises a spring element that retains the shape predetermined by the manufacturer.

Accordingly, one of the objectives of the present invention is to create an apparatus for the dynamic stabilization of bones or bone fragments, in particular vertebrae, with at least one longitudinal support that can be fixed to the vertebrae and can effortlessly be adapted to the most diverse situations for implantation, with no impairment of the dynamics.

This objective is achieved by the characterizing features given in claim 1, preferred structural details of which are described in the subordinate claims. The basic idea of the present invention is thus that the at least one longitudinal support, which for example is fixed between two adjacent pedicle screws, is so constructed that by applying a predetermined bending force, it can be deformed plastically from a first shape state “A” into a second, alternative shape state “B”, the bending force needed for this purpose being distinctly greater than the peak forces that occur in vivo. While remaining in each of the two stable shape states, however, the longitudinal support should be flexible within the limits imposed by the mechanical interaction between fixation system and vertebral-column segment, which define a so-called “elastic flexion range”.

It should be noted at this juncture that the apparatus in accordance with the invention is fundamentally also suitable for anterior implantation, when it is desired to shift the center of rotation of the affected spinal-column segment toward the anterior.

An especially advantageous embodiment of the apparatus in accordance with the invention solves the problem of bending into shape a longitudinal support made of a biocompatible high-performance plastic, in that a metal rod is disposed centrally in the support. The metal rod must on one hand be so thin that its critical bending angle is larger than or equal to the maximal angle through which the stabilized vertebrae will bend when connected to the dynamic fixation system, while on the other hand being thick enough that the longitudinal support retains the shape into which it was bent in situ.

To obtain a particular bending elasticity it is conceivable for the central metal rod to be coated with several layers, each of which is distinguished from the others by having a modulus of elasticity related in a very special way to those of the other layers.

The patent DE 93 08 770 U1 describes a plastic rod with a metal core. This plastic rod serves a trial rod or template that can be used to adapt the shape of the longitudinal support optimally to the position and orientation of the pedicle screws. For this purpose it must be possible to adjust the shape of the trial rod by hand in situ, in the patient. Accordingly, the trial rod is made of a soft plastic (e.g., silicone) and a metal rod that can easily be plastically deformed (e.g., of pure aluminum). If the trial rod has the same outside diameter as the longitudinal support, the trial rod exactly reproduces the shape that is necessary for a stress-free seating of the support in the pedicle screws.

The present invention is distinguished from the teaching according to DE 93 08 770 U1 on the basis of the condition, specified above, that
a) the at least one longitudinal support can be deformed plastically from a first shape state “A” into a second, alternative shape state “B” by applying a predetermined bending force, the bending force needed for this purpose being distinctly greater than the peak forces that occur in vivo, and
b) the at least one longitudinal support is, however, flexible while in each of the two stable shape states, specifically within the limits imposed by the mechanical interaction between fixation system and vertebral-column segment, which define a so-called “elastic flexion range”.

Preferably the bending elasticity of the longitudinal support in accordance with the invention is specified such that when fixed at one end, the support can be elastically deflected through an angle of 5° to 12°, in particular about 8°, while remaining in a dimensionally stable state.

In order to initiate the above-mentioned pain alleviation and healing processes, the at least one longitudinal support must be so configured that it is as stiff as possible with respect to the compression and shear forces encountered in vivo, and that the construction consisting of longitudinal support plus anchoring means is substantially torsion-proof.

The longitudinal support in accordance with the invention can
a) be shaped like a flat band or strip, or
b) have a cross section that is rotationally symmetric, circular, polygonal or elliptical, and that may remain constant over the entire length of the longitudinal support or else can vary according to a mathematically describable rule and/or change in a stepwise manner.

Furthermore, care should be taken that the longitudinal support is dimensioned such that in the above-mentioned “elastic flexion range” its surface tension is always below the dynamic fracture limit. This applies in particular also to the individual components of a longitudinal support that consists of a core enclosed in a covering layer or layers.

When the at least one longitudinal support made of biocompatible plastic is so designed that it has the same geometry as the metallic longitudinal supports normally used for fusions, then the dynamic fixation system can at any time be converted to a fusion-inducing fixation system, inasmuch as the dynamic longitudinal support can be replaced by a metallic (and correspondingly stiff) longitudinal support with no need to exchange the pedicle screws, and conversely.

It is also intended to make available a dynamic stabilization system based on the following fundamental considerations:

In the present case the aim is to develop a dynamic pedicle-screw system that can be inserted posteriorly and does not cause pathologically altered spinal-column segments to become fused, but rather is specifically designed to support the function of the affected structures.

As mentioned at the outset, primary indications for a dynamic system are diseases, inflammations and/or injuries in the region of the intervertebral disc, the ligament apparatus, the facet joints and/or the subchondral bone. In these situations it is important to modify the load pattern in the affected region in such a way that the pathological state at least does not become worse. The ideal would be healing, although in the case of degenerative diseases, at least, this is unlikely to be possible.

However, the aim of the dynamic system to be developed is not only to preserve the present pathological state or even to bring about healing, but also to combine with the affected structures so as to form a unit that enhances the structures' metabolism.

As soon as a pedicle-screw system is put into place from the posterior aspect, the center of rotation of the affected movement segment is shifted posteriorly out of the intervertebral disk, however flexible the support system may be. However, a backward shift of the center of rotation as far as the region of the posterior facet joints can have the following effects, depending on the pathology:

1. Pain source “posterior facet joints:

Depending on the position of the posteriorly shifted center of rotation relative to the posterior facet joints and on the axial compressibility of the system, the movement in the joints is more or less drastically reduced. This creates the prerequisite for a degeneratively altered joint to be able to recover, inasmuch as the missing hyaline joint cartilages are, at least theoretically, replaced by fibrous cartilages (the Passive Motion Principle of Salter). However, the prerequisite for recovery is also that the system can be inserted without stress.

2. Pain source “posterior annulus” of the intervertebral disk, lordosis and disk height preserved:

In the posterior annulus fissures can appear because of traumatic developments or degenerative modifications. These fissures often start on the nuclear side and progressively penetrate toward the outer, innervated edge of the annulus. With Magnetic Resonance Imaging (MRI) it is possible to identify pockets of fluid in the region of such fissures. These so-called “hot spots” can be an indication of an inflammatory process in the region of the posterior annulus. Inflammations can occur, for instance, in the region where granulation tissue is growing in from the exterior and/or where nerve endings, which can also come from the interior, encounter nuclear material being pressed through fissures in the annulus (physiological pain). This inflammatory process is promoted in the long term by the continuously maintained flow of nuclear material. Theoretically, however, an inflammation is not absolutely necessary to produce pains; instead, the mechanical pressure exerted by a pocket of liquid on afferent nerve endings can in itself cause pain. A suitable stabilization can stop the inflammatory process and even induce healing. In this regard the following considerations are relevant:

Because of the posterior displacement of the center of rotation of the spinal segment, its range of movement in both flexion and extension is drastically reduced, and the axial force acting on the intervertebral disk is uniformly distributed over the whole disk. As a result, during “global” flexion/extension of the patient the nuclear material is no longer being squeezed back and forth; that is, less of the nuclear material that triggers the inflammatory process is pressed through fissures in the posterior annulus and toward the site of inflammation. This is the situation required for the inflammation to become healed so that a repair process can begin.

3. Problem of “primary disk hernia”:

In the case of disk hernia there is a connection between the nucleus and the vicinity of the annulus. Therefore nuclear material can continuously flow through annular fissures. During nucleotomy the material that has emerged is removed along with material taken from the nucleus, the latter in order to avoid secondary disk hernia. In this process, the lesion in the posterior annulus is enlarged by the surgery.

Here, again, a posterior shift of the center of rotation of the spinal segment reduces the subsequent flow of nuclear material. The disk hernia cannot continue to increase, and emerging material that had not already been surgically removed becomes encapsulated and is resorbed by the body. A repair process can take place at the posterior annulus.

Thus for cases of primary disk hernia a dynamic system at least theoretically offers the advantage that the surgical intervention can be minimized (there is no need for opening of the epidural space or for additional damage to the annulus). Thus optimal conditions can be created for healing of the disk and restoration of its function.

4. Pain source “posterior annulus of the intervertebral disk” (collapsed disk):

The pain in the posterior annulus can be caused by delamination of the annulus. Delamination of the posterior annulus occurs when the nucleus becomes dehydrated and therefore the disk collapses. Shifting the center of rotation to a more posterior position, in the region behind the posterior facet joints, reduces the pressure in the region of the posterior annulus, which inhibits further delamination of the posterior annulus. This creates the prerequisites for the annulus to heal or form a cicatrix—assuming, of course, that the annulus has the necessary healing potential.

5. Pain source “cover plate/subchondral bone”:

MRI makes it possible to observe changes in the fluid balance within the subchondral bones of the vertebrae. In particular, it is also possible to detect a sclerotic change in the bony cover plate indicating that the nutrient supply to the intervertebral disk has encountered a bottleneck or been completely interrupted. A sclerotic alteration of the cover plate can hardly be reversed: the degenerative “devastation” of the disk is preprogrammed.

It is also conceivable for the fluid content to be increased. For this there are two explanations:
a) inflammation in the subchondral region, which causes inflammatory pain.
b) accumulation because the connecting channels in the bony cover plate of the vertebra have become “stopped up” (owing to sclerotic alterations, etc.).

The first of these, inflammation, can be alleviated by suitable means insofar as the affected tissue is not permanently damaged.

In the second case, at least in theory the increased pressure in the subchondral bone resulting from the stoppage can cause mechanical stimulation of the afferent nerve endings (mechanical pain). Measures taken to reduce the pressure in the subchondral region can at least reduce the mechanical pain, if not make it vanish altogether. In this case, however, the cause of the problem is very difficult to eliminate.

The posterior shifting of the center of rotation in the region behind the posterior facet joints reduces the load not only on the intervertebral disk, but also on the underlying subchondral bone. Thus with a suitable dynamic fixation the prerequisites for alleviation of pain are created and even for healing, in the case of inflammation in the region of the subchondral bone.

6. Pain source “nerve root”:

Mechanical pressure on the nerve root produces an insensitivity radiating into the lower extremities as well as muscle weakness, but not pain. Pains (sciatica, etc.) arise only when inflammation-inducing nuclear material emerges through fissures in the posterior annulus and presses on the nerve roots.

Here, again, a posterior shift of the center of rotation of the spinal segment reduces the flow of nuclear material that stimulates the inflammatory process. This creates the prerequisites for the inflammation to heal, so that a repair process can to some extent be initiated at the posterior annulus. It is even conceivable for a disk hernia to be reversed if no new nuclear material flows out.

7. Problem of “spinal-column fracture”:

In the case of spinal-column fracture the structures usually affected are the vertebra situated cranially in the relevant segment and the associated intervertebral disk. With the good blood perfusion allowed by the present-day fixation techniques as described above, healing of the bone tissue in the vertebra no longer presents a problem. Healing of the disk, in contrast, follows other rules because of the inadequate blood flow and takes significantly longer. If after ca. 6 months a stiff posterior fixation is converted to a flexible posterior fixation, this relieves the load on the disk and permits certain movement components. Depending on the degree of load relief and the remaining extent of movement, the prerequisites for healing of the disk are satisfied—assuming that the supply to the disk from the subchondral region of the adjacent vertebra is not disturbed (for example, by callus formation in the region of the subchondral bone).

The posterior shift of the center of rotation of the associated spinal segment brought about by a posteriorly inserted dynamic system reduces the load on the traumatized intervertebral disk, as has been described above, and furthermore allows an axial deformation that is important for the nutrition of the disk.

In the light of the preceding considerations, it is also the goal of the present invention, by moving the center of rotation of an affected spinal segment to a more posterior position, to immobilize the posterior annulus of the affected intervertebral disk, with the consequence that posterior emergence of nuclear material is correspondingly reduced while an amount of axial deformation that is important for the nutrition of the disk simultaneously remains possible; this is done in such a way that pressure is largely homogeneously exerted on the disk and the associated cover plates. Accordingly, it is also an objective to make available a sufficiently dynamic stabilization system, through which the center of rotation of the affected spinal segment is shifted posteriorly in a predetermined manner.

The system in accordance with the invention should thus also be distinguished on one hand by an extremely elegant construction and surgical technique as well as the advantages of a dynamic system, and on the other hand by offering the possibility of optimally determining the posterior center of rotation of a prespecified spinal-column segment.

This objective is achieved in accordance with the invention by the characteristics given in claim 13, both independently of the considerations underlying claims 1 to 12 and also, in particular, in combination therewith.

That is, from a medical viewpoint it can certainly be advantageous for the bone-anchoring means, such as pedicle screws, to comprise openings or slots to receive the longitudinal support that can be positioned at an axial distance from the opposed distal end that is variable, in particular adjustable, so that the longitudinal support itself can be positioned at a correspondingly variable distance from the vertebra. As a result, for example, the posterior center of rotation can be adjusted to suit the individual. The simplest embodiment of these considerations consists in having a supply of pedicle screws with screw heads of different heights, in which the slots to receive the longitudinal support are formed. An alternative design comprises screw heads that can be moved into different axial positions on the shaft of the pedicle screw; in this case, for example, the screw heads can be screwed onto the screw shafts and individually fixed at different heights by means of locknuts.

It is also conceivable to make available pedicle screws with separate screw heads that can be stuck onto the threaded shaft and that have openings of different lengths to receive the longitudinal support. In this case it should be kept in mind that after a pedicle screw has been put into place, it need not subsequently be set lower or higher (with the danger of loosening) to ensure that the longitudinal support will be disposed at the prescribed distance from the vertebra. All that is needed is to exchange the screw head, or to alter its height.

In the following an exemplary embodiment of a stabilization system in accordance with the invention is explained in greater detail with reference to the attached drawings, wherein

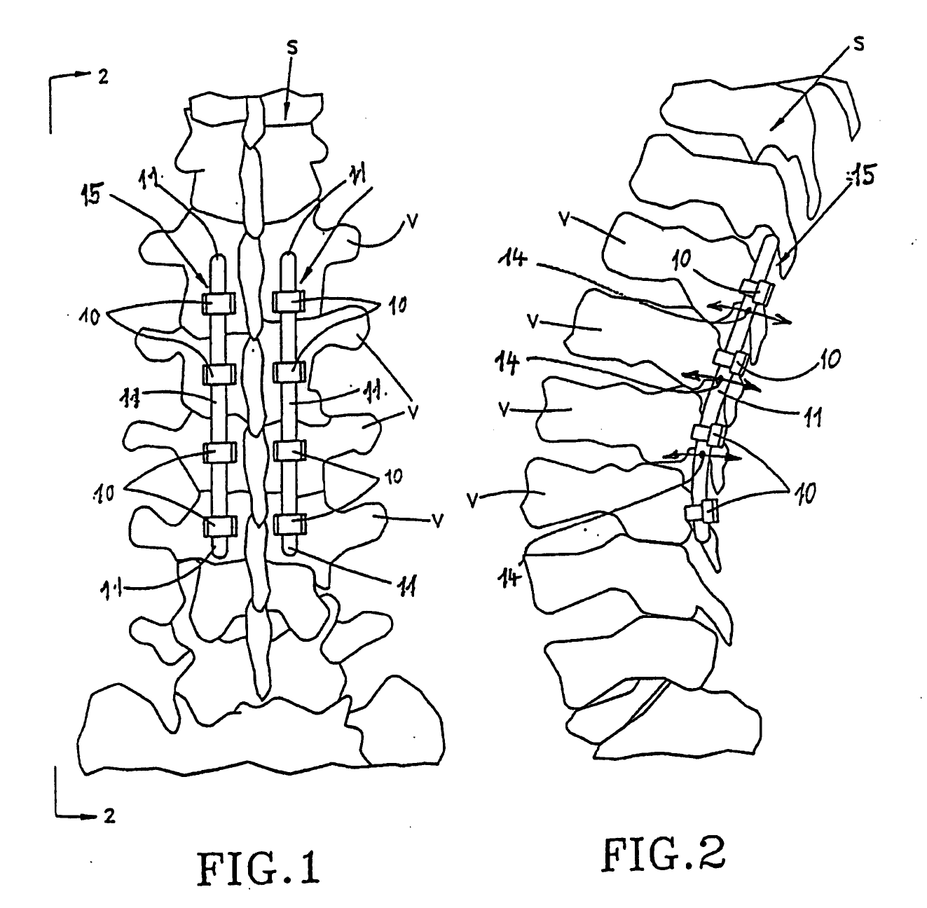

FIG. 1 shows a spinal segment comprising four vertebrae, with posterior stabilization of this segment as seen from posterior;

FIG. 2 shows the arrangement according to FIG. 1 in side view along line 2-2 in FIG. 1; and

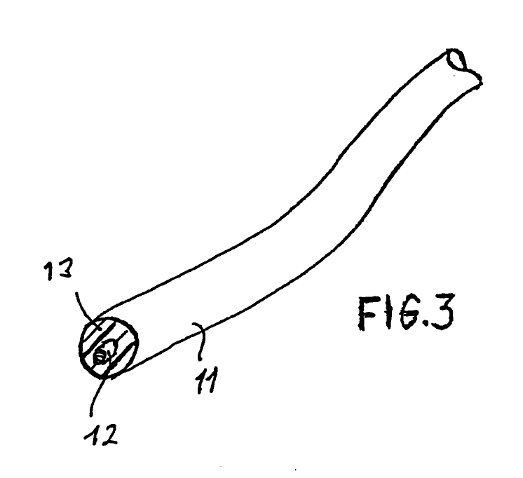

FIG. 3 shows a longitudinal support constructed in accordance with the invention in the shape of a round rod, partly in section, partly in perspective, and at an enlarged scale.

In FIGS. 1 and 2 is shown part of a spinal column, wherein the individual vertebrae are identified by the reference letters “V”. The spinal column is identified by the letter “S”.

The individual vertebrae “V” are stabilized posteriorly, for which purpose pedicle screws are screwed from the back into four vertebrae “V”. Each of the screw heads comprises openings or slots to receive a rod-shaped longitudinal support 11. The longitudinal support 11, as FIG. 3 also shows particularly well, is constructed in the shape of a round rod and is fixed in place by clamping in the heads of the pedicle screws 10. In this way a spinal segment comprising four vertebrae “V” can be stabilized. The longitudinal support or supports 11 is/are so designed as to be plastically deformable by application of a prespecified bending force, so that they are changed from a first stable shape state into a second, alternative stable shape state as shown in FIGS. 1 and 2. While in this implantation state, however, the longitudinal supports 11 are intended to be flexible within prespecified limits, as was presented in the introductory section. This achieves a dynamic stabilization of a predetermined spinal segment, with all the advantages explained above.

Specifically, in the embodiment presented here the longitudinal support 11 is provided with a core 12 made of metal, in particular titanium or a titanium alloy, encased in a human-tissue-compatible plastic 13. The plastic deformability of the longitudinal support 11 is ensured primarily by the metallic core 12, whereas the flexibility in the deformed state is determined primarily by the plastic casing 13. The above-mentioned bending elasticity of the longitudinal support 11 is indicated in FIG. 2 by a double-headed arrow 14. It is sufficient that when the longitudinal support 11 is clamped at one end, it can be elastically deflected by an angle of 5° to 12°, in particular about 8° (double-headed arrow 14), while remaining in a dimensionally stable state.

It should also be mentioned at this juncture that the apparatus described here can comprise connecting means for the longitudinal support, which can be used to connect at least two support sections together. The support-connecting means can, for example, comprise two oppositely situated openings or slots to serve as support receptacles, into each of which one end section of a longitudinal support can be inserted and fixed by a clamping screw or the like.

The support-connecting means can be made either rigid or, preferably, flexible. They allow supports to be implanted one segment at a time, and permit extremely individual stabilization of a section of the spinal column.

In can also be seen in FIGS. 1 and 2 that the stabilization of a spinal-column section by means of the apparatus in accordance with the invention is always carried out in such a way that flexibility is available only in the context of flexion and extension. Thus pressure on the cover plate and intervertebral disk is considerably reduced, with no impairment of the axial deformation of the disk, which is important for its nutrition.

The longitudinal support thus described must of course also be designed such that it can be permanently deformed with a prespecified force, which is greater than the peak forces encountered anatomically, i.e. in vivo. This deformation is carried out apart from the implantation, and preferably should be possible without the need for special accessory devices. The deformation is carried out “on site” by the surgeon.

In both the long direction of the longitudinal support and also the transverse direction, the support should be stable, i.e. unyielding, with respect to the anatomically customary shear forces. Furthermore, it is very often desirable for the longitudinal support to be stable with respect to torsion, in order to ensure that the affected vertebral segment extends, as a rule approximately horizontally, substantially only around a posteriorly shifted center of rotation. As already mentioned above, the longitudinal support can be constructed as a flat band or strip. In the embodiment described here, supports in the shape of a round rod are implanted.

With regard to the bending elasticity of the longitudinal support in accordance with the invention it should also be mentioned that the angular range cited above refers to a length of the support 11 that corresponds to the spacing of two adjacent vertebrae, i.e. to a distance of about 2-6 cm, in particular about 4-5 cm.

In other respects, regarding preferred embodiments, reference is made to those according to claims 16-18, which state for example that the core can be shaped as a flat band or strip, with a width that is the same as or smaller than the corresponding dimension of the longitudinal support. This configuration is naturally primarily appropriate for supports that have a band-like shape.

The width and/or height of the band-like core can vary continuously or stepwise along the length of the longitudinal support, at least over one longitudinal section thereof.

Regarding a rotationally symmetrical core, reference is made to claim 17.

In particular, it is fundamentally also conceivable for the diameter of the core to become continuously larger or smaller, at least in sections, so that the core acquires the form of a wedge or cone. A stepwise change in the core diameter is also conceivable, although in this last case the transitions in the regions of a step are preferably rounded in order to reduce or completely avoid the stresses associated with steps.

Alternatively, it is also conceivable to form a groove in the region of a stepwise transition, in order to reduce stresses.

All the characteristics disclosed in the application documents are claimed as essential to the invention insofar as they are new to the state of the art individually or in combination.

### LIST OF REFERENCE NUMERALS

10 Pedicle screw
11 Longitudinal support
12 Core
13 Plastic casing
14 Double-headed arrow
15 Stabilization system
S Spinal column
V Vertebra

1. Apparatus for the dynamic stabilization of bones or bone fragments, in particular spinal vertebrae (V), with at least one longitudinal support (11) that can be fixed to the vertebrae (V), characterized in that the at least one longitudinal support (11) is so constructed that by application of a prespecified bending force it can be plastically deformed from a first stable shape state “A” into a second, alternative stable shape state “B”, but while in the first as well as in the second state remains flexible within predetermined limits (“elastic flexion range”).

2. Apparatus according to claim 1, characterized in that the longitudinal support (11) is such that when clamped at one end, while within a stable shape state “A” or “B” it can be elastically deflected by an angle of 5° to 12°, in particular about 8°, over a length corresponding to the spacing of two adjacent vertebrae, or about 2 to 5 cm.

3. Apparatus according to claim 1, characterized in that the longitudinal support (11) is constructed so as to be stable, i.e. unyielding, both with respect to anatomically usual longitudinal shear forces and with respect to anatomically usual transverse shear forces.

4. Apparatus according to claim 1, characterized in that the longitudinal support (11) is constructed so as to be substantially stable with respect to torsion.

5. Apparatus according to claim 1, characterized in that the longitudinal support (11) is constructed in the shape of a flat band or strip.

6. Apparatus according to claim 1, characterized in that the longitudinal support (11) is constructed so as to be rotationally symmetrical.

7. Apparatus according to claim 1, characterized in that the longitudinal support (11) is hollow, in particular is constructed as a hollow rod.

8. Apparatus according to claim 1, characterized in that the longitudinal support (11) comprises an in particular plastically deformable core (12) made of metal, in particular titanium or a titanium alloy, which is encased in a human-tissue-compatible plastic (13), in particular one that ensures flexibility within a stable shape state.

9. Apparatus according to claim 1, characterized in that the longitudinal support (11) is so dimensioned that within the elastic flexion range its surface stress is always below the dynamic breaking stress.

10. Apparatus according to claim 8, characterized in that in the case of a longitudinal support with core (12), both the core and the casing (13) are dimensioned such that in the elastic flexion range the surface stress of both core (12) and casing (13) is always below the respective dynamic breaking stress.

11. Apparatus according to claim 8, characterized in that the core (12) is encased in more than one layer.

12. Apparatus according to claim 1, characterized in that it comprises bone-anchoring means, in particular pedicle screws (10), to which the longitudinal support or supports (11) can be fixed.

13. Apparatus according to claim 1, characterized in that it comprises longitudinal-support-connecting means, which can be used to connect at least two support sections to one another.

14. Apparatus according to claim 13, characterized in that the longitudinal-support-connecting means comprise two oppositely situated support-receiving openings, into each of which an end section of the support can be inserted and fixed by way of a clamping screw or similar clamping element.

15. Apparatus according to claim 1, characterized in that the bone-anchoring means comprise longitudinal-support-receiving openings that can be spaced at variable axial distances from the opposite distal end, so that the longitudinal support (11) can be adjusted to a correspondingly different distance from the vertebra (V).

16. Apparatus according to claim 8, characterized in that the core (12) is constructed in the form of a flat band or strip, with a width smaller than or equal to the corresponding dimension of the longitudinal support.

17. Apparatus according to claim 8, characterized in that the core (12) is rotationally symmetrical, in particular circular, with either a constant diameter or a diameter that varies along the length of the longitudinal support.

18. Apparatus according to claim 17, characterized in that the diameter of the core (12), at least in sections, is continually enlarged or reduced and/or altered in a stepwise manner, such that in the last case the transitions in the region of a step are constructed so as to reduce stress, in particular are rounded.
